(19) United States
(12) Patent Application Publication
(10) Pub. No.: US 2026/0220685 A1
(43) Pub. Date: Jul. 30, 2026

(54) APPLICATION RECOMMENDATION SYSTEM AND METHOD, STORAGE MEDIUM, AND VEHICLE

(71) Applicant: FAURECIA (CHINA) HOLDING Co., Ltd., Shanghai (CN)

(72) Inventors: Wulong ZHANG, Shanghai (CN); Tudui HONG, Shanghai (CN); Qingyang SU, Shanghai (CN); Tianjiao WANG, Shanghai (CN)

(73) Assignee: FAURECIA (CHINA) HOLDING Co., Ltd., Shanghai (CN)

(21) Appl. No.: 19/457,268

(22) Filed: Jan. 23, 2026

(30) Foreign Application Priority Data
Jan. 26, 2025 (CN) 202510125717.7

(51) Int. Cl. G06Q 30/0601; A61B 5/0205; A61B 5/024; A61B 5/08; A61B 5/1455; A61B 5/16; A61B 5/18; G07C 5/02

(52) U.S. Cl. CPC G06Q 30/0631; A61B 5/0205; A61B 5/14551; A61B 5/165; A61B 5/18; G07C 5/02; A61B 5/024; A61B 5/0816; A61B 2503/22

(57) ABSTRACT

An application recommendation system and method, a storage medium, and a vehicle are disclosed in the present disclosure, which relate to the technical field of vehicle. The application recommendation system includes: an acquisition module, configured to acquire application determination data, where the application determination data includes driving advice data and health data of a target person in the vehicle, the driving advice data is generated from driving behavior data of a driver of the vehicle; a processing module, configured to determine a recommended application according to the application determination data; and a display module, configured to display the recommended application.

### CROSS-REFERENCE TO RELATED APPLICATION(S)

The present disclosure is filed based on Chinese Patent Application with the application No. 202510125717.7, filed on Jan. 26, 2025, and claims priority to the Chinese Patent Application, the entire content of the Chinese Patent Application is incorporated herein by reference.

### TECHNICAL FIELD

The present disclosure relates to the technical field of vehicle, and in particular, to an application recommendation system and method, a storage medium, and a vehicle.

### BACKGROUND

With the increasing level of vehicle intelligence, vehicles may provide users with more service experiences (such as music, navigation, or communication, etc.) during driving. The above service experiences are mainly achieved through applications, which means that before using the services provided by the vehicle, corresponding application needs to be installed in the vehicle in advance.

### SUMMARY

In a first aspect, an application recommendation system applied to an automotive application store is provided, which includes: an acquisition module, configured to acquire application determination data, where the application determination data includes driving advice data and health data of a target person in the vehicle, the driving advice data is generated from driving behavior data of a driver of the vehicle; a processing module, configured to determine a recommended application according to the application determination data; and a display module, configured to display the recommended application.

In one implementation, the processing module is specifically configured to: determine a plurality of sets of candidate applications according to the application determination data, and perform weighting processing on initial application scores of respective candidate applications in the plurality of sets of candidate applications, so as to obtain target application scores of the respective candidate applications; and determine a candidate application whose target application score meets an application determination condition as the recommended application.

In one implementation, the processing module is specifically configured to: input the application determination data into application determination models of a plurality of application determination models respectively, so as to obtain a plurality of sets of candidate applications; where an application determination model is used to output a set of candidate applications, and the plurality of application determination models are trained based on different training data.

In one implementation, the application determination data includes numerical-type data and character-type data, and the processing module is specifically configured to: determine a first feature value of the numerical-type data and a second feature value of the character-type data; where first feature value is extracted by performing normalization and standardization on the numerical-type data, and the second feature value is a binary feature value obtained by performing one hot coding on the character-type data; and input the first feature value and the second feature value into the application determination models of the plurality of application determination models respectively, so as to obtain the plurality of sets of candidate applications. In one implementation, the acquisition module is specifically configured to: acquire the health data of the target person according to a cockpit monitoring system; where the health data includes one or more of: a gender, an age, a heart rate, a respiratory rate, a blood oxygen saturation, or a mood.

In one implementation, the application determination data further includes one or more of: the driving behavior data of the driver inside the vehicle; an application that has been installed on the vehicle; application data of an application that has not been installed on the vehicle in an application platform; a location of the vehicle and meteorological data for the location.

In a second aspect, an application recommendation method applied to an automotive application store is provided, and the method includes: acquiring application determination data, where the application determination data comprises driving advice data and health data of target person in a vehicle, where the driving advice data is generated from driving behavior data of a driver of the vehicle; determining a recommended application according to the application determination data; and displaying the recommended application. In one implementation, determining the recommended application according to the application determination data includes: determining a plurality of sets of candidate applications according to the application determination data, and performing weighting processing on initial application scores of respective candidate applications in the plurality of sets of candidate applications, so as to obtain target application scores of the respective candidate applications; and determining a candidate application whose target application score meets an application determination condition as the recommended application.

In one implementation, determining the plurality of sets of candidate applications according to the application determination data includes: inputting the application determination data into application determination models of a plurality of application determination models respectively, so as to obtain a plurality of sets of candidate applications; where an application determination model is used to output a set of candidate applications, and the plurality of application determination models are trained based on different training data.

In one implementation, the application determination data includes numerical-type data and character-type data, and determining the plurality of sets of candidate applications according to the application determination data includes: determining a first feature value of the numerical-type data and a second feature value of the character-type data; where first feature value is extracted by performing normalization and standardization on the numerical-type data, and the second feature value is a binary feature value obtained by performing one hot coding on the character-type data; and inputting the first feature value and the second feature value into the application determination models of the plurality of application determination models respectively, so as to obtain the plurality of sets of candidate applications.

In a third aspect, an application recommendation apparatus applied to an automotive application store is provided, and the apparatus includes an acquisition unit, a determination unit, and a display unit.

The acquisition unit is configured to acquire application determination data, where the application determination data includes driving advice data and health data of a target person in the vehicle, the driving advice data is generated from driving behavior data of a driver of the vehicle. The determination unit is configured to determine a recommended application according to the application determination data. The display module is configured to display the recommended application. In one implementation, the determination unit is specifically configured to: determine a plurality of sets of candidate applications according to the application determination data, and perform weighting processing on initial application scores of respective candidate applications in the plurality of sets of candidate applications, so as to obtain target application scores of the respective candidate applications; and determine a candidate application whose target application score meets an application determination condition as the recommended application.

In one implementation, the determination unit is specifically configured to: input the application determination data into application determination models of a plurality of application determination models respectively, so as to obtain a plurality of sets of candidate applications; where an application determination model is used to output a set of candidate applications, and the plurality of application determination models are trained based on different training data.

In one implementation, the application determination data includes numerical-type data and character-type data, and the determination unit is specifically configured to: determine a first feature value of the numerical-type data and a second feature value of the character-type data; where first feature value is extracted by performing normalization and standardization on the numerical-type data, and the second feature value is a binary feature value obtained by performing one hot coding on the character-type data; and input the first feature value and the second feature value into the application determination models of the plurality of application determination models respectively, so as to obtain the plurality of sets of candidate applications, where an application determination model is used to output a set of candidate applications, and any two of the plurality of application determination models are trained based on different training data.

In a fourth aspect, an electronic device is provided, which includes a memory and a processor. The memory and the processor are coupled. The memory is configured to store computer program codes, and the computer program codes includes computer instructions. When the processor executes the computer instructions, the electronic device is caused to perform the method according to the second aspect.

In a fifth aspect, a computer-readable storage medium is provided, and the computer-readable storage medium stores computer instructions. When the computer instructions are executed on an electronic device, the electronic device is caused to perform the method according to the second aspect.

### BRIEF DESCRIPTION OF THE DRAWINGS

FIG. 1 is a structural schematic diagram of an architecture of an application recommendation system provided in the embodiments of the present disclosure.

FIG. 2 is a schematic diagram for collecting application determination data provided in the embodiments of the present disclosure.

FIG. 3 is a first schematic diagram for displaying a recommended application provided in the embodiments of the present disclosure.

FIG. 4 is a second schematic diagram for displaying a recommended application provided in the embodiments of the present disclosure.

FIG. 5 is a schematic diagram for recommending an application provided in the embodiments of the present disclosure.

FIG. 6 is a flowchart of a download process of a recommended application provided in the embodiments of the present disclosure.

FIG. 7 is a first schematic diagram of a determination process of a recommended application provided in the embodiments of the present disclosure.

FIG. 8 is a flowchart of a model training process shown in an exemplary embodiment provided in the embodiments of the present disclosure.

FIG. 9 is a second schematic diagram of a determination process of a recommended application shown in an exemplary embodiment provided in the embodiments of the present disclosure.

FIG. 10 is a third schematic diagram of a determination process of a recommended application shown in an exemplary embodiment provided in the embodiments of the present disclosure.

FIG. 11 is a block diagram of an application recommendation apparatus shown in an exemplary embodiment provided in the embodiments of the present disclosure.

FIG. 12 is a block diagram of an electronic device shown in an exemplary embodiment provided in the embodiments of the present disclosure.

### DETAILED DESCRIPTION

The technical solutions in the embodiments of the present disclosure will be described with combination of the accompanying drawings in the embodiments of the present disclosure below.

In the description of the present disclosure, unless otherwise specified, a character “/” means an “or” relationship, for example, “A/B” may represent A or B. “And/or” in the present disclosure is just an association relationship that describes relevant objects, indicating that three relationships can exist. For example, A and/or B may mean these three situations: A exists alone; A and B exist simultaneously; or B exist alone. In addition, “at least one” or “multiple/a plurality of/the plurality of” refers to that a number of objects is two or more. The wordings such as “first,” “second,” etc., do not limit quantity or an execution order, and the wordings such as “first,” “second,” etc., do not limit that corresponding objects are different, either.

Different users have varying application requirements for vehicle infotainment systems, but interfaces of the existing vehicle infotainment systems often fail to intelligently match user requirements, requiring users to perform tedious operations to find the desired applications. It can be seen that a current manner for application installation is time-consuming.

Addressing the above-mentioned technical problems, an application recommendation system applied to an automotive application store is provided in the embodiments of the present disclosure. The application recommendation system includes: an acquisition module, configured to acquire application determination data, where the application determination data includes driving advice data and health data of a target person in the vehicle, the driving advice data is generated from behavior parameters of a person driving the vehicle; a processing module, configured to determine a recommended application according to the application determination data; and a display module, configured to display the recommended application.

In this way, the driving advice data and the health data of the target person inside the vehicle are acquired. Furthermore, the recommend application is determined according to the driving behavior and the health data of the target person. In this way, the corresponding recommended application may be determined and displayed according to users with different states without the need for manual operation by the users, thus reducing the time for the users to search for and interact with the applications.

As shown in FIG. 1, FIG. 1 shows an application recommendation system 100 applied to an automotive application store provided in the embodiments of the present disclosure, which includes an acquisition module 101, a processing module 102, and a display module 103. The processing module 102 is respectively connected to the acquisition module 101 and the display module 103 for communication.

In some embodiments, the application recommendation system 100 is applied to in an on-board terminal. The on-board terminal may be an electronic device.

The acquisition module 101 is configured to acquire application determination data.

Herein, the application determination data includes driving advice data and health data of target person in a vehicle, and the driving advice data is generated from behavior data of a person driving the vehicle.

In some embodiments, the acquisition module 101 is configured to acquire the application determination data while the vehicle is running.

In other embodiments, the acquisition module 101 is configured to acquire the application determination data in response to an application recommendation operation.

In some embodiments, the acquisition module 101 is configured to acquire the health data of the target person according to a cockpit monitoring system. The health data includes one or more of: a gender, an age, a heart rate, a respiratory rate, a blood oxygen saturation, or a mood.

For example, the target person is a driver. The acquisition module 101 is configured to acquire the health data of the driver according to a driver monitoring system (DMS). As another example, the target person is a passenger. The acquisition module 101 is configured to acquire the health data of the passenger according to an occupancy monitoring system (OMS).

In some embodiments, the acquisition module 101 is configured to acquire the health data of the target person from a health database.

In the embodiments of the present disclosure, the mood may be textual data. For example, the mood may be anger, disgust, fear, happiness, sadness, or surprise.

In the embodiments of the present disclosure, the driving advice data may include a vehicle driving advice and/or a mood relief advice.

In some embodiments, the processing module 102 is configured to generate the driving advice data according to acceleration information, driving speed information, and braking information of the vehicle. For example, in a case where an acceleration of the vehicle is greater than a preset acceleration, the processing module 102 generates a vehicle driving advice text of “slowing acceleration”. As another example, in a case where the acceleration of the vehicle is greater than the preset acceleration, and a number of sudden braking times is greater than a preset number, the processing module 102 is configured to generate a mood relief advice text of “settling mood”.

It should be noted that the preset acceleration and the preset number are pre-configured by an operation and maintenance person, and may also be determined according to a driving environment of the vehicle, which are not limited in the embodiments of the present disclosure.

In another scenario, the processing module 102 is configured to generate the driving advice data according to the health data of the target person in the health database.

In the embodiments of the present disclosure, the application determination data may further include one or more of: the driving behavior data of the driver inside the vehicle; application data of an application that has been installed on the vehicle; application data of an application that has not been installed on the vehicle in an application platform; a location of the vehicle and meteorological data for the location.

Herein, the driving behavior data may include one or more of: a steering frequency, a steering amplitude, a degree of opening of an accelerator pedal, a degree of opening of a brake pedal, or a driving speed. The application data of the application that has been installed may include one or more of: an online duration, an application type, a usage frequency, or an installation duration of the installed application. The application data of the application that has not been installed may include one or more of: a number of downloads, an application rating, an application category, and an application name.

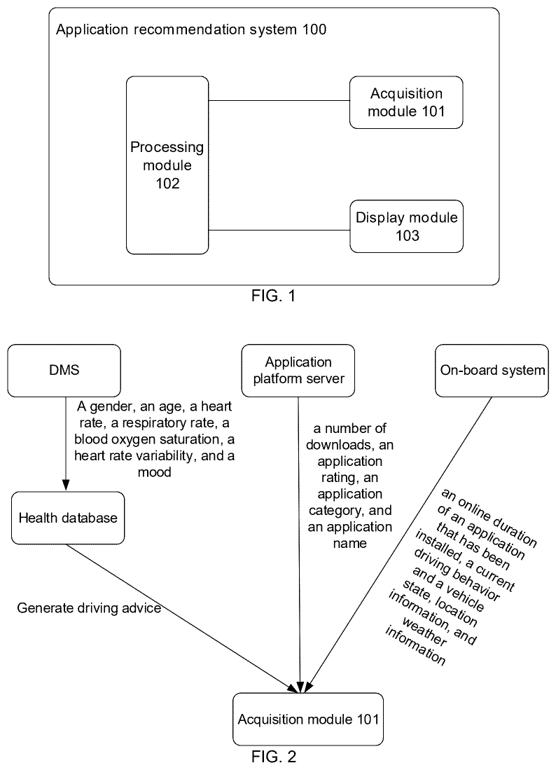

In some embodiments, as shown in FIG. 2, FIG. 2 shows a schematic diagram for collecting the application determination data. In FIG. 2, the acquisition module 101 acquires the application data of the application that has not been installed sent from an application platform server, including a number of downloads, an application rating, an application category, and an application name. The DMS may store received health data in the health database. The health data includes: a gender, an age, a heart rate, a respiratory rate, a blood oxygen saturation, and a mood of a person in the vehicle. Furthermore, the health database generates a driving advice according to the health data of the target person and sends the driving advice to the acquisition module 101. The acquisition module 101 acquires an online duration of an application that has been installed, current driving behavior data and a vehicle state, historical behavior data, location information, and weather information collected by the an on-board system of the vehicle.

In some embodiments, the acquisition module 101 is configured to acquire on-board original data, and acquires the application determination data according to the on-board original data. For example, the acquisition module 101 is configured for on-board locating data, on-board DMS data, and the health data in the health database.

For example, the acquisition module 101 is configured to input the on-board locating data into a location determination model, and output a location of the vehicle. As another example, the acquisition module 101 is configured to input the on-board DMS data into an information determination model, and output an age and a gender.

It should be noted that the location determination model and the information determination model are pre-configured by an operation and maintenance person.

It can be understood that the acquisition module 101 is configured to acquire five dimensions of information: a user profile, a user habit and preference, a user health state, a user driving habit, and application market information.

The processing module 102 is configured to determine an recommended application according to the application determination data.

In some embodiments, the processing module 102 is configured to determine a recommended application type according to the application determination data, and to determine the recommended application according to the recommended application type.

In some embodiments, the processing module 102 is configured to determine a plurality of sets of candidate applications according to the application determination data, and perform weighting processing on respective candidate applications of the plurality of sets of candidate applications, to obtain application scores of the respective candidate applications. The processing module 102 is further configured to determine a candidate application whose application score meets an application determination condition as the recommended application.

In some embodiments, the processing module 102 is configured to input the application determination data into application determination models of a plurality of application determination model respectively, so as to obtain the plurality of sets of candidate applications. Herein, an application determination model is used to output a set of candidate applications, and any two of the plurality of application determination models are trained based on different training data.

In some embodiments, the processing module 102 is configured to perform normalization and standardization on numerical-type data in the application determination data, so as to obtain processed numerical-type data, and determine a first feature value of the processed numerical-type data. Furthermore, the processing module 102 is configured to determine one hot code of character-type data in the application determination data, and determine a second feature value of the one hot code. Subsequently, the processing module 102 is further configured to: input the first feature value and the second feature value into the application determination models of the plurality of application determination models respectively, so as to obtain the plurality of sets of candidate applications.

In some embodiments, the processing module 102 is further configured to pre-process data in the application determination data. For example, the processing module 102 is further configured to input the application determination data into a pre-processing model to obtain application determination data from removing a missing value and a abnormal value.

It should be noted that the pre-processing model is pre-configured by the operation and maintenance person.

The display module 103 is configured to display the recommended application.

In the embodiments of the present disclosure, displaying the recommended application may be displaying an identifier of the recommended application. The identifier of the recommended application is used to launch or install the recommended application. For example, the identifier of the recommended application may include an application icon, link information, etc.

In some embodiments, the processing module 102 is configured, in a case where the recommended application is determined, to acquire a download link of the recommended application in the application platform according to a name of the recommended application, and generate an identifier of the recommended application according to the download link in the application platform. Furthermore, the processing module 102 is configured to indicate the display module 103 to display the identifier of the recommended application. Correspondingly, the display module 103 is configured to display the identifier of the recommended application. In this way, the application recommendation system may download and install the recommended application in response to users clicking on the identifier of the recommended application, making it easier for a user to use and thus improving the user experience.

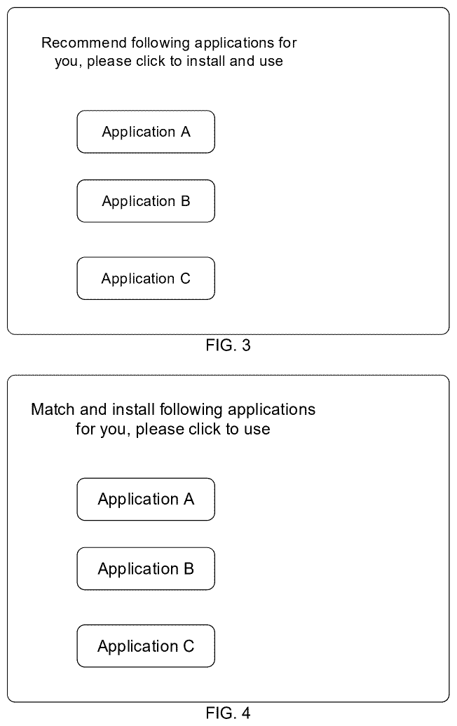

For example, as shown in FIG. 3, the display module 103 is configured to display an identifier of application A, an identifier of application B, and an identifier of application C, and an installation prompt message “Recommend following applications for you, please click to install and use”.

In some embodiments, the processing module 102 is configured, in a case where the recommended application is determined, to download the recommended application from the application platform according to a name of the recommended application, and install the recommended application. Furthermore, the processing module 102 is configured to indicate the display module 103 to display the identifier of the recommended application. Correspondingly, the display module 103 is configured to display the identifier of the recommended application. In this way, the application recommendation system may launch the recommended application in response to users clicking on the identifier of the recommended application, making it easier for a user to use and thus improving the user experience.

For example, as shown in FIG. 4, the display module 103 is configured to display the identifier of application A, the identifier of application B, and the identifier of application C, and an installation prompt message “Match and install following applications for you, please click to use”.

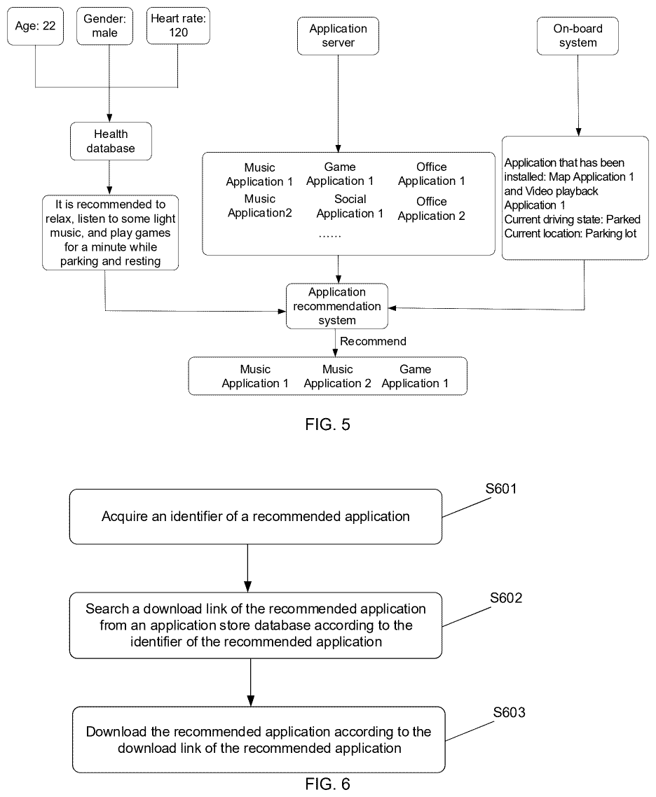

In order to better understand an application recommendation process of the application recommendation system 100 provided in the embodiments of the present disclosure, as shown in FIG. 5, a schematic diagram of application recommendation is shown in FIG. 5. User data collected in the health database includes: “gender: male, age: 22, and heart rate: 120”. Herein, the user data is acquired through a DMS camera. Furthermore, according to the user data from the health database, a driving advice text of “It is recommended to relax, listen to some light music, and play games for a minute while parking and resting.” is obtained. A plurality of types of applications are obtained from an application server: application type 1: music application 1 and music application 2; application type 2: game application 1 and social application 1; application type 3: office application 1 and office application 2, etc. According to the on-board system, the following vehicle information is obtained: installed applications: map application 1 and video playback application 1; current driving state: parked; current location: parking lot. Finally, the application recommendation system 100 recommends three applications according to the vehicle information, the plurality of types of applications, and the driving advice text: music application 1, music application 2, and game application 1. Herein, these three applications are obtained through voting by the application recommendation system 100. In this way, the application icons of the three recommended applications are displayed, and the three recommended applications are associated with their corresponding download links, so that a user is capable of installing and using them by clicking on a download button.

In some embodiments, a download process of a recommended application is shown in FIG. 6, which includes S601-S603.

In S601, an identifier of a recommended application is acquired.

In S602, a download link of the recommended application is searched from an application store database according to the identifier of the recommended application.

In S603, the recommended application is downloaded according to the download link of the recommended application.

The application recommendation system 100 provided in the embodiments of the present disclosure brings at least the following beneficial effects: the recommended application is determined according to the driving behavior and the health data of the target person. In this way, the corresponding recommended application may be determined according to users with different states without the need for manual operation by the users, thus reducing the time for the users to search for and interact with the applications.

In some embodiments, in order to acquire applications more accurately, the processing module is specifically configured to: determine a plurality of sets of candidate applications according to the application determination data, and perform weighting processing on initial application scores of respective candidate applications in the plurality of sets of candidate applications, so as to obtain target application scores of the respective candidate applications; and further determine a candidate application whose target application score meets an application determination condition as the recommended application.

In the embodiments of the present disclosure, an initial application score is a pre-configured value. For example, the initial application score may be 6, or may be 10, which is not limited in the embodiments of the present disclosure. Initial application scores for different candidate applications may be the same or different, which is not limited in the embodiments of the present disclosure.

In the embodiments of the present disclosure, the application determination condition may be an application score being greater than or equal to a preset application score, or an application score ranking being greater than or equal to a preset threshold, which is not limited in the embodiments of the present disclosure.

It should be noted that the preset application score is pre-configured by the operation and maintenance person.

In some embodiments, the processing module 102 is configured to input the application determination data into application determination models of a plurality of application determination model respectively, so as to obtain the plurality of sets of candidate applications. An application determination model is used to output a set of candidate applications, and the plurality of application determination models are trained based on different training data.

In some embodiments, the processing module 102 is configured to: determine a feature value of the application determination data, and input the feature value of the application determination data into application determination models of a plurality of application determination model respectively, so as to obtain the plurality of sets of candidate applications. Furthermore, the processing module 102 is configured to: perform weighting processing on initial application scores of the respective candidate applications in the plurality of sets of candidate applications, so as to obtain target application scores of the respective candidate applications. Furthermore, the processing module 102 is configured to determine a candidate application whose target application score meeting an application determination condition as the recommended application.

In some embodiments, the processing module 102 is configured to perform normalization and standardization on numerical-type data in the application determination data, so as to obtain processed numerical-type data, and determine a first feature value of the processed numerical-type data. Furthermore, the processing module 102 is configured to perform one hot coding on the character-type data, to obtain a second feature value, where the second feature value is a binary eigenvalue. Subsequently, the processing module 102 is configured to: input the first feature value and the second feature value into the application determination models of the plurality of application determination models respectively, so as to obtain the plurality of sets of candidate applications. It should be noted that weights corresponding to the respective sets of candidate applications may be the same or different, which is not limited in the embodiments of the present disclosure.

It can be understood that the plurality of sets of candidate applications are determined according to the application determination data, and the initial application scores of the respective candidate applications are weighted, so as to obtain the recommended application according to the target application scores of the candidate applications. In this way, the recommended application is selected from the plurality of candidate applications, and the recommended applications may be obtained more accurately.

In one design, in order to determine a matched recommended application, the processing module 102 is specifically configured to: input the application determination data into application determination models of a plurality of application determination model respectively, so as to obtain the plurality of sets of candidate applications.

An application determination model for the application determination data to input in the embodiments of the present disclosure is a trained application determination model.

In some embodiments, the application determination model may be a neural network model, a combination prediction algorithm model, or a support vector machine model, etc., which is not limited in the embodiments of the present disclosure. For example, the application determination model may be a non-linear multi-module decision model.

In some embodiments, the plurality of application determination models may be models in the same type, or may be models in different types, which are not limited in the embodiments of the present disclosure.

In some embodiments, a number of the plurality of application determination models is not limited in the embodiments of the present disclosure, which may be 3, 5, or 2. In addition, there is no limit to a number of applications for each set of candidate applications, which may be 3, 4, or 2.

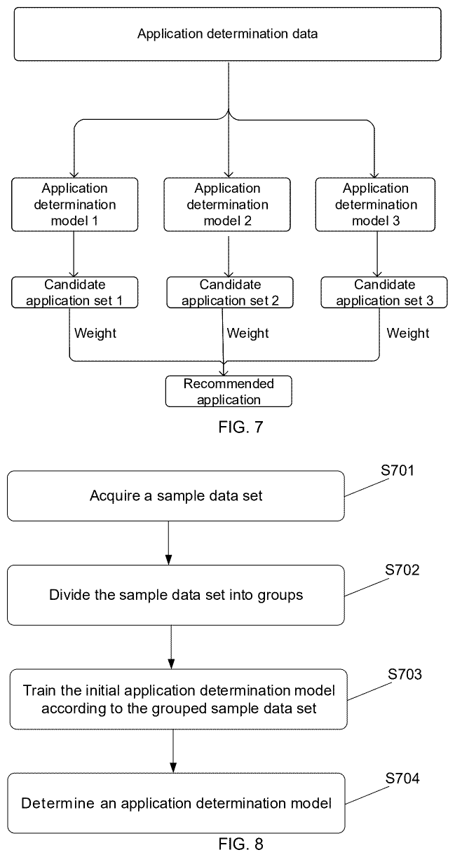

Exemplarily, as shown in FIG. 7, FIG. 7 shows an example of the plurality of application determination models being three application determination models. The application determination data is input into three application determination models: application determination model 1, application determination model 2, and application determination model 3, so as to obtain three sets of candidate applications: candidate application set 1, candidate application set 2, and candidate application set 3. Furthermore, respective candidate applications in the three sets of candidate applications are weighted, and the weighted candidate applications are voted on, so as to obtain the recommended application.

Before inputting the application determination data into the plurality of application determination models, an initial application determination model is trained in the embodiments of the present disclosure, so as to obtain the plurality of application determination models.

Specifically, as shown in FIG. 8, FIG. 8 shows a flowchart of a model training process, which includes S701-S704.

In S701, a sample data set is acquired.

In some embodiments, each sample data in the sample data set includes: an age, a gender, health data, driving behavior data, application data of an application that has been installed on the vehicle, application data of an application that has not been installed on the vehicle in an application platform, a location, a weather, and a calibrated application.

In S702, the sample data set is divided into groups.

In some embodiments, the sample data set is divided into groups according to a number of initial application determination models. For example, if the number of the initial application determination models is 3, the sample data set may be divided into 3 groups. As another example, if the number of the initial application determination models is 4, the sample data set may be divided into 4 groups.

Furthermore, the grouped sample data is classified to obtain: a training sample data group and a validation sample data group. For example, two groups of sample data are obtained: sample data group 1 and sample data group 2. Furthermore, the sample data group 1 is configured as the training sample data group, and the sample data group 2 is configured as the validation sample data group. In this way, an initial application determination model 1 is trained using the sample data group 1, and the trained initial application determination model 1 is validated using sample data group 2. Further, the set sample data group 2 is configured as the training sample data group, and the sample data group 1 is configured as the validation sample data group. In this way, an initial application determination model 2 is trained using the sample data group 2, and the trained initial application determination model 2 is validated using sample data group 1.

It should be noted that a number of sample data in each sample data group may be the same or different, and the number of sample data in each sample data group is not limited in the embodiments of the present disclosure.

In S703, the initial application determination model is trained according to the grouped sample data set.

Specifically, the initial application determination model is trained using the training sample data group, and model parameters of the initial application determination model are optimized using the gradient descent and the loss function. Subsequently, the initial application determination model is validated using the validation sample data group. If a loss value of the loss function tends to stabilize, module optimization is stopped.

For example, taking the initial application determination model being a non-linear initial model as an example, in a case of configuring three non-linear initial models: non-linear initial model 1, non-linear initial model 2, and non-linear initial model 3, each non-linear initial model is trained and validated separately according to its training sample data group and validation sample data group, so as to obtain a trained non-linear model and a corresponding weight.

In S704, an application determination model is determined.

It can be understood that by inputting the application determination data into different models to obtain the plurality of sets of candidate applications, and determining the recommended application from the plurality of sets of candidate applications, the accuracy of application recommendation may be improved.

In one design, in order to quickly obtain a recommendation result, the processing module 102 in the embodiments of the present disclosure is specifically configured to: perform normalization and standardization on numerical-type data in the application determination data, so as to obtain processed numerical-type data, and determine a first feature value of the processed numerical-type data. One hot code of character-type data in the application determination data is determined, and a second feature value of the one hot coding is determined. The first feature value and the second feature value are input into the application determination models of the plurality of application determination models respectively, so as to obtain the plurality of sets of candidate applications.

In some embodiments, the processing module 102 classifies the application determination data, to obtain the numerical-type data and the character-type data. Furthermore, the processing module 102 performs normalization and standardization on the numerical-type data, and performs feature extraction on the processed numerical-type data to obtain the first feature value.

For example, normalization and standardization is performed on the numerical-type data such as age, heart rate, respiratory rate, and temperature in the application determination data.

In the embodiments of the present disclosure, in order to avoid the significant influence of a certain value on the application determination model, a deviation Min-Max normalization formula is used to scale data to the same scale. The deviation normalization formula is shown in Formula 1 below.

$${X}^{′} = \frac{X - min ( X )}{max ( X ) - min ( X )} \qquad Formula ( 1 )$$

Herein, X′ is normalized data, X is any data in the numerical-type data before normalization, min (X) is the smallest value in the numerical-type data, and max (X) is the largest value in the numerical-type data.

In the embodiments of the present disclosure, in order to facilitate feature extraction, data is also standardized. For example, the data is converted to a standard normal distribution with a mean of 0 and a standard deviation of 1. The standardization formula is as shown in Formula 2 below.

$${X}^{′} = \frac{X - μ}{σ} \qquad Formula ( 2 )$$

Herein, X′ is the standardized data, X is any data in the numerical-type data before normalization, σ is the standard deviation of all data in the numerical-type data, and μ is the mean of all data in the numerical-type data.

In some embodiments, the processing module 102 is configured to input the pre-processed application determination data into a preset feature extraction model, and output a binary data feature. Furthermore, the processing module 102 is configured to input the binary data feature into the application determination model, and output a serial number of the recommended application, and obtain a link of the recommended application according to the serial number of the recommended application.

In addition, the processing module 102 is configured to determine a one hot code of the character-type data, and determine a second feature value of the one hot code. In this way, for non numerical-type data, it is necessary to use a manner of one hot coding to replace the non numerical-type data with a binary vector for use in the model, accelerating data processing and obtaining recommended applications more quickly.

For example, the one hot code of character-type data (such as the mood, location, a driving advice text, and an application type in the application determination data) is determined.

In some embodiments, before performing normalization and standardization on the application determination data, the processing module 102 is further configured to pre-process original application determination data.

For example, pre-processing includes missing processing and abnormality processing.

For missing processing: in a case where the processing module 102 detects that certain data in the application determination data is missing, preset data corresponding to the missing data is used for filling.

In some embodiments, in a case where the processing module 102 detects that original data corresponding to a certain parameter value is missing, the original data corresponding to the parameter value is deleted, and a preset value corresponding to the parameter value is determined as a target value corresponding to the parameter value.

For abnormality processing: in a case where the processing module 102 detects that certain data in the application determination data is abnormal data, a non-linear regression algorithm is used to perform abnormality processing on an abnormal value that is too large or too small, thus to obtain rationalized data.

It can be understood that data pre-processing is used to clean and transform original data, in order to improve data quality.

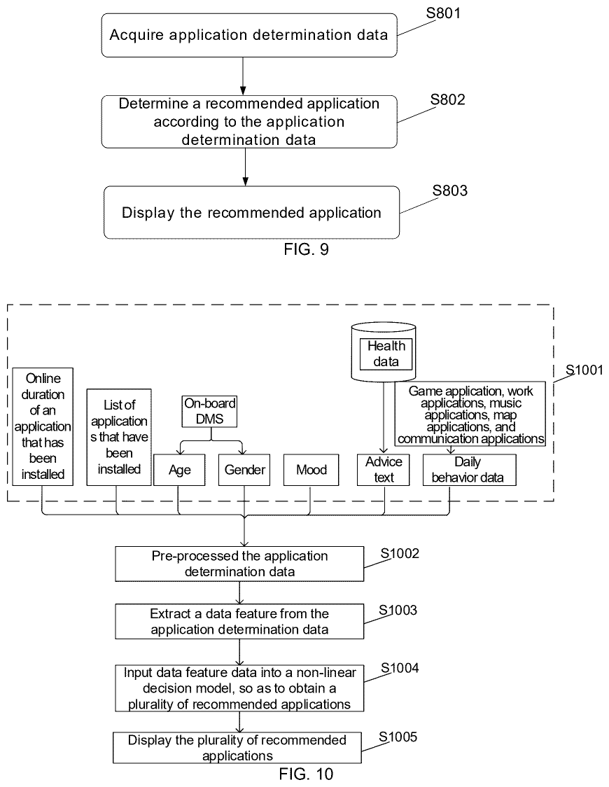

In a design, in order to reduce the time for users to search for and interact with applications, a flowchart of an application recommendation method applied to an automotive application store is provided in the embodiments of the present disclosure, as shown in FIG. 9, the method includes S801-S803.

In S801, application determination data is acquired.

Herein, the application determination data includes driving advice data and health data of target person in a vehicle, and the driving advice data is generated from behavior parameters for driving the vehicle.

In some embodiments, an electronic device acquires the application determination data in response to an application recommendation instruction. Acquisition for parameters of the application determination data in this step may refer to an acquisition manner described for the above-mentioned acquisition module 101, which will not be repeated herein.

In S802, a recommended application is determined according to the application determination data.

As an implementation, the electronic device determines the recommended application according to the application determination data and application determination models.

In some embodiments, the electronic device pre-processes the application determination data in a case where the application determination data is acquired to obtain the pre-processed data, and extracts a data feature of the pre-processed data. Furthermore, the electronic device inputs the data feature into an application determination model to obtain a recommended application.

In some embodiments, the electronic device determines a plurality of sets of candidate applications according to the application determination data. Weighting processing is performed on respective candidate applications in the plurality of sets of candidate applications to obtain application scores of the respective candidate applications. Furthermore, the electronic device determines a candidate application whose target application score meeting an application determination condition as the recommended application.

In some embodiments, the electronic device inputs the application determination data into application determination models of a plurality of application determination model respectively, so as to obtain the plurality of sets of candidate applications.

In some embodiments, the electronic device performs normalization and standardization on numerical-type data in the application determination data, so as to obtain processed numerical-type data, and determine a first feature value of the processed numerical-type data. Furthermore, one hot code of character-type data in the application determination data is determined, and a second feature value of the one hot code is determined. Subsequently, the electronic device inputs the first feature value and the second feature value into the application determination models of the plurality of application determination models respectively, so as to obtain the plurality of sets of candidate applications.

Data pre-processing, feature extraction, and determination of the recommended application in this step may refer to the data processing manner described for the above-mentioned processing module 102, which will not be repeated herein.

In another scenario, the application determination data includes health data and a mood. The electronic device determines a current state of a target person according to the health data and the mood of the target person, and obtains the recommended applications according to the current state of the target person and a target correspondence. The target correspondence includes a plurality of states and recommended applications corresponding to the respective states.

In S803, the recommended application is displayed.

Displaying the recommended application in this step may refer to a displaying manner described for the above-mentioned display module 102, which will not be repeated herein.

In one design, the above-mentioned S802 includes S8021-S8023.

In S8021, a plurality of sets of candidate applications are determined according to the application determination data.

In S8022, weighting processing is performed on initial application scores of the respective candidate applications in the plurality of sets of candidate applications, so as to obtain target application scores of the respective candidate applications.

In S8023, a candidate application whose target application score meets an application determination condition is determined as the recommended application.

In one design, the above-mentioned S8021 includes S901.

In S901, the application determination data is input into application determination models of a plurality of application determination models respectively, so as to obtain a plurality of sets of candidate applications.

In one design, the above-mentioned S8021 includes S902-S905.

In S902, normalization and standardization are performed on numerical-type data in the application determination data, so as to obtain processed numerical-type data.

In S903, a first feature value of the processed numerical-type data.

In S904, a second feature value is obtained by performing one hot coding on the character-type data.

Herein, the second feature value is a binary feature value.

In S905, the first feature value and the second feature value are input into the application determination models of the plurality of application determination models respectively, so as to obtain the plurality of sets of candidate applications.

In order to better understand the application recommendation method provided in the embodiments of the present disclosure, as shown in FIG. 10, a flowchart of an application recommendation method is shown, which includes S1001-S1005.

In S1001, application determination data is acquired.

Specifically, as shown in FIG. 10, the application determination data shown in FIG. 10 includes: an online duration of an application that has been installed, a list of applications that have been installed, an age, a gender, a mood, an advice text, and daily behavior data. Herein, in FIG. 10, the age and the gender are obtained according to an on-board DMS; the advice text is obtained according to health data in the health database; the daily behavior data is obtained according to game applications, work applications, music applications, map applications, and communication applications.

In S1002, the application determination data is pre-processed.

In S1003, a data feature is extracted from the application determination data.

In S1004, data feature data is input into a non-linear decision model, so as to obtain a plurality of recommended applications.

In S1005, the plurality of recommended applications are displayed.

The solutions provided in the embodiments of the present disclosure are introduced above from the perspective of methods. In order to implement the above functions, the control apparatus includes corresponding hardware structures and/or software modules for implementing various functions. Those skilled in the art should easily realize that the present disclosure can be implemented in the form of hardware or a combination of hardware and computer software in combination with units and algorithm steps of examples described in the embodiments disclosed by the description. Whether a certain functionality is implemented by hardware or by computer software driving hardware depends on the specific application and design constraint conditions of the technical solutions. A professional technician may use different methods to implement the described functions for each specific application, but such implementation should not be considered beyond the scope of the present disclosure.

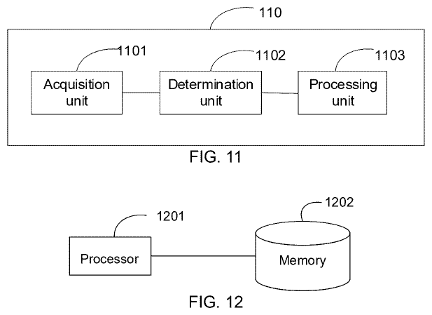

FIG. 11 shows an application recommendation apparatus according to an exemplary embodiment. As shown in FIG. 11, the application recommendation apparatus 110 includes an acquisition unit 1101, a determination unit 1102, and a display unit 1103.

The acquisition unit 1101 acquires application determination data.

Herein, the application determination data includes driving advice data and health data of target person in a vehicle, and the driving advice data is generated from behavior parameters for driving the vehicle.

The determination unit 1102 determines a recommended application according to the application determination data.

The display unit 1103 displays the recommended application.

Herein, the recommended application is used to launch or install the recommended application.

In one implementation, the determination unit 1102 is specifically configured to: determine a plurality of sets of candidate applications according to the application determination data, and perform weighting processing on initial application scores of respective candidate applications in the plurality of sets of candidate applications, so as to obtain target application scores of the respective candidate applications; and determine a candidate application whose target application score meets an application determination condition as the recommended application.

In one implementation, the determination unit 1102 is specifically configured to: input the application determination data into application determination models of a plurality of application determination models respectively, so as to obtain a plurality of sets of candidate applications; where an application determination model is used to output a set of candidate applications, and the plurality of application determination models are trained based on different training data.

In one implementation, the application determination data includes numerical-type data and character-type data, and the determination unit is specifically configured to: determine a first feature value of the numerical-type data and a second feature value of the character-type data, where the is obtained from extraction with performing normalization and standardization on the numerical-type data; and input the first feature value and the second feature value into the application determination models of the plurality of application determination models respectively, so as to obtain the plurality of sets of candidate applications, where an application determination model is used to output a set of candidate applications, and any two of the plurality of application determination models are trained based on different training data.

In one implementation, the determination unit 1102 is specifically configured to: perform one hot coding on the character-type data to obtain the second feature value; where the second feature value is a binary feature value.

FIG. 12 shows a block diagram of an electronic device according to an exemplary embodiment. As shown in FIG. 12, the electronic device includes, but not limited to: a processor 1201 and a memory 1202.

Herein, the above-mentioned memory 1202 is configured to store instructions that are executable for the above-mentioned processor 1201. It can be understood that the above-mentioned processor 1201 is configured to execute instructions, so as to implement the application recommendation method in the above-mentioned embodiments.

It should be noted that those skilled in the art may understand that structures of the electronic device shown in FIG. 12 do not constitute a limitation on the electronic device. The electronic device may include more or fewer components than those shown in FIG. 12, or certain components may be combined, or components may be arranged in different ways.

The processor 1201 is a control center of the electronic devices, which connects various parts of the entire electronic device through various interfaces and circuits. By running or executing software programs and/or modules stored in the memory 1202, and calling data stored in the memory 1202, the processor 1201 may perform various functions of the electronic device and process data, thereby monitoring the electronic device as a whole. The processor 1201 may include one or more processing units. In some embodiments, the processor 1201 may integrate an application processor and a modulation and demodulation processor, where the application processor mainly handles operating systems, user interfaces, and application programs, and the modulation and demodulation processor mainly handles wireless communication. It can be understood that the above-mentioned modulation and demodulation processor may not be integrated into the processor 1201.

The memory 1202 may be configured to store software programs and various data. The memory 1202 may mainly include an area for storing programs and an area for storing data, where the area for storing programs may store application programs required by an operating system, or at least one functional module (such as a determination unit, a processing unit, etc.), etc. In addition, the memory 1202 may include a high-speed random access memory, and may further include a non-volatile memory (e.g., at least one disk storage component, flash memory component, or other volatile solid-state storage components).

In an exemplary embodiment, a computer-readable storage medium including instructions is further provided, such as the memory 1202 including the instructions, and the above-mentioned instructions may be executed by the processor 1201 of the electronic device, so as to implement the methods described in the above-mentioned embodiments.

In some embodiments, the computer-readable storage medium may be a non-transitory computer-readable storage medium, for example, the non-transitory computer-readable storage medium may be a read-only memory (ROM), a random access memory (RAM), a CD-ROM, a magnetic tape, a floppy disk, an optical data storage device, etc.

In an exemplary embodiment, a computer program product including one or more instructions is further provided in the embodiments of the present disclosure, and the one or more instructions may be executed by the processor 1201 of the electronic device, so as to implement the methods described in the above-mentioned embodiments.

It should be noted that when the instructions in the computer-readable storage medium or one or more instructions in the computer program product are executed by the processor of the electronic device, the various processes of the above method embodiments may be implemented, and the same technical effects as the above methods may be achieved, which will not be repeated herein to avoid repetition.

Through the description of the above implementations, those of ordinary skill in the art may clearly understand that for the convenience and simplicity of the description, the division of the above functional modules is given merely as an example. In practical applications, the above functions can be allocated to different functional modules according to requirements, that is, the internal structure of the apparatus may be divided into different functional modules to complete all or part of the functions described above.

In the several embodiments provided in the present disclosure, it should be understood that the disclosed apparatuses and methods may be implemented in other ways. For example, the apparatus embodiments described above are only illustrative. For example, the division of modules or units is only a logical functional division. In actual implementation, there may be other division methods, for example, multiple units or components can be combined or integrated into another apparatus, or some features can be ignored or not performed. On the other hand, the coupling or direct coupling or communicative connection with each other as shown or discussed may be indirect coupling or communicative connection of apparatus or units via some interfaces, which may be electrical, mechanical, or in other forms.

The units described as separate components may or may not be physically separated, and the component(s) shown as units may be one physical unit or multiple physical units, that is, may be located in one place, or may be distributed to multiple different places. A part or all of the units may be selected according to actual needs, to implement the purpose of the solutions of the embodiments.

In addition, the functional units in the embodiments of the present disclosure may be integrated into a single processing unit or the functional units may exist physically and separately, or two or more units may be integrated into a unit. The above integrated unit may be implemented in the form of hardware, or may be implemented in the form of a software functional unit.

If the integrated unit is implemented in the form of the software functional unit and sold or used as an independent product, the integrated unit may be stored in a readable storage medium. Based on this understanding, the technical solutions of the present disclosure may be essentially, or a part of the technical solutions of the present disclosure that contributes to the prior art may be, all or part of the technical solutions may be, embodied in the form of a software product, and the software product is stored in a storage medium, which includes several instructions to enable a device (which may be a single-chip microcomputer, a chip, etc.) or a processor to perform all or part of steps of methods in various embodiments of the present disclosure. The aforementioned storage media include: a U disk, a mobile hard disk, a ROM, a RAM, a magnetic disk or an optical disk and other media that may store program codes.

The above descriptions are merely specific implements of the present disclosure, but the scope of protection of the present disclosure is not limited thereto, and any variations or replacements within the technical scope disclosed in the present disclosure shall fall within the protection scope of the present disclosure. Therefore, the protection scope of the present application should be subject to the protection scope of the claims.

What is claimed is:

1. An application recommendation system, applied to an automotive application store, the system comprising:
an acquisition module, configured to acquire application determination data, wherein the application determination data comprises driving advice data and health data of target person in a vehicle, wherein the driving advice data is generated from driving behavior data of a driver of the vehicle;
a processing module, configured to determine a recommended application according to the application determination data; and
a display module, configured to display the recommended application.

2. The system according to claim 1, wherein the processing module is further configured to:
determine a plurality of sets of candidate applications according to the application determination data, and perform weighting processing on initial application scores of respective candidate applications in the plurality of sets of candidate applications, so as to obtain target application scores of the respective candidate applications; and
determine a candidate application whose target application score meets an application determination condition as the recommended application.

3. The system according to claim 2, wherein the processing module is further configured to:
input the application determination data into application determination models of a plurality of application determination models respectively, so as to obtain the plurality of sets of candidate applications; wherein an application determination model is used to output a set of candidate applications, and the plurality of application determination models are trained based on different training data.

4. The system according to claim 3, wherein the application determination data comprises numerical-type data and character-type data, and the processing module is further configured to:
determine a first feature value of the numerical-type data and a second feature value of the character-type data; wherein the first feature value is obtained from extraction with performing normalization and standardization on the numerical-type data, and the second feature value is a binary feature value obtained by performing one hot coding on the character-type data; and
input the first feature value and the second feature value into the application determination models of the plurality of application determination models respectively, so as to obtain the plurality of sets of candidate applications.

5. The system according to claim 1, wherein the acquisition module is further configured to:
acquire the health data of the target person according to a cockpit monitoring system;
wherein the health data comprises one or more of: a gender, an age, a heart rate, a respiratory rate, a blood oxygen saturation, or a mood.

6. The system according to claim 1, wherein the application determination data further comprises one or more of:
the driving behavior data of the driver inside the vehicle;
an application that has been installed on the vehicle;
application data of an application that has not been installed on the vehicle in an application platform;
a location of the vehicle and meteorological data for the location.

7. An application recommendation method, applied to an automotive application store, wherein the method comprises:
acquiring application determination data, wherein the application determination data comprises driving advice data and health data of target person in a vehicle, wherein the driving advice data is generated from driving behavior data of a driver of the vehicle;
determining a recommended application according to the application determination data; and
displaying the recommended application.

8. The method according to claim 7, wherein determining the recommended application according to the application determination data comprises:
determining a plurality of sets of candidate applications according to the application determination data, and performing weighting processing on initial application scores of respective candidate applications in the plurality of sets of candidate applications, so as to obtain target application scores of the respective candidate applications; and
determining a candidate application whose target application score meets an application determination condition as the recommended application.

9. The method according to claim 8, wherein determining the plurality of sets of candidate applications according to the application determination data comprises:
inputting the application determination data into application determination models of a plurality of application determination models respectively, so as to obtain the plurality of sets of candidate applications; wherein an application determination model is used to output a set of candidate applications, and the plurality of application determination models are trained based on different training data.

10. The method according to claim 9, wherein the application determination data comprises numerical-type data and character-type data, and determining the plurality of sets of candidate applications according to the application determination data comprises:
determining a first feature value of the numerical-type data and a second feature value of the character-type data; wherein the first feature value is obtained from extraction with performing normalization and standardization on the numerical-type data, and the second feature value is a binary feature value obtained by performing one hot coding on the character-type data; and
inputting the first feature value and the second feature value into the application determination models of the plurality of application determination models respectively, so as to obtain the plurality of sets of candidate applications.

11. The method according to claim 7, wherein acquiring the health data of the target person comprises:
acquiring the health data of the target person according to a cockpit monitoring system; wherein the health data comprises one or more of: a gender, an age, a heart rate, a respiratory rate, a blood oxygen saturation, or a mood.

12. The method according to claim 7, wherein the application determination data further comprises one or more of:
the driving behavior data of the driver inside the vehicle;
an application that has been installed on the vehicle;
application data of an application that has not been installed on the vehicle in an application platform;
a location of the vehicle and meteorological data for the location.

13. A non-transitory computer-readable storage medium, wherein when computer execution instructions stored on the computer-readable storage medium being executed by a processor of a processing apparatus, the processing apparatus is capable of performing the method according to claim 7.

14. An electronic device, comprising: a processor and a memory, wherein the memory stores instructions executable for the processor; the processor is configured, upon the instructions being executed by the processor, to enable the electronic device to:
acquire application determination data, wherein the application determination data comprises driving advice data and health data of target person in a vehicle, wherein the driving advice data is generated from driving behavior data of a driver of the vehicle;
determine a recommended application according to the application determination data; and
display the recommended application.

15. The electronic device according to claim 14, wherein the electronic device is further enabled to:
determine a plurality of sets of candidate applications according to the application determination data, and perform weighting processing on initial application scores of respective candidate applications in the plurality of sets of candidate applications, so as to obtain target application scores of the respective candidate applications; and
determine a candidate application whose target application score meets an application determination condition as the recommended application.

16. The electronic device according to claim 15, wherein the electronic device is further enabled to:
input the application determination data into application determination models of a plurality of application determination models respectively, so as to obtain the plurality of sets of candidate applications; wherein an application determination model is used to output a set of candidate applications, and the plurality of application determination models are trained based on different training data.

17. The electronic device according to claim 16, wherein the application determination data comprises numerical-type data and character-type data, and wherein the electronic device is further enabled to:
determine a first feature value of the numerical-type data and a second feature value of the character-type data; wherein the first feature value is obtained from extraction with performing normalization and standardization on the numerical-type data, and the second feature value is a binary feature value obtained by performing one hot coding on the character-type data; and
input the first feature value and the second feature value into the application determination models of the plurality of application determination models respectively, so as to obtain the plurality of sets of candidate applications.

18. The electronic device according to claim 14, wherein the electronic device is further enabled to:
acquire the health data of the target person according to a cockpit monitoring system;
wherein the health data comprises one or more of: a gender, an age, a heart rate, a respiratory rate, a blood oxygen saturation, or a mood.

19. The electronic device according to claim 14, wherein the application determination data further comprises one or more of:
the driving behavior data of the driver inside the vehicle;
an application that has been installed on the vehicle;
application data of an application that has not been installed on the vehicle in an application platform;
a location of the vehicle and meteorological data for the location.
